(19) United States
(12) Patent Application Publication
(10) Pub. No.: US 2018/0125373 A1
(43) Pub. Date: May 10, 2018

(54) SYSTEM AND METHOD FOR REMOVING BACKGROUND NOISE FROM PHOTOACOUSTIC IMAGE

(71) Applicant: SOGANG UNIVERSITY RESEARCH FOUNDATION, Seoul (KR)

(72) Inventors: Jin Ho CHANG, Seoul (KR); Heamin Kim, Incheon (KR)

(73) Assignee: SOGANG UNIVERSITY RESEARCH FOUNDATION, Seoul (KR)

(21) Appl. No.: 15/802,735

(22) Filed: Nov. 3, 2017

(30) Foreign Application Priority Data
Nov. 4, 2016 (KR) 10-2016-0146551

(51) Int. Cl. A61B 5/00; A61B 8/08; G06T 5/00

(52) U.S. Cl. CPC A61B 5/0095; A61B 8/481; G06T 2207/10132; G06T 5/002; A61B 5/7203

(57) ABSTRACT

Disclosed is a method of removing a background noise from a photoacoustic image, the method including applying an ultrasonic signal to a target absorbing body having a multi-modal microbubble contrast agent injected thereinto and receiving the ultrasonic signal reflected by the target absorbing body in order to acquire an ultrasonic image, when a photoacoustic signal is generated from the target absorbing body having the multi-modal microbubble contrast agent injected thereinto as the result of absorbing a laser pulse applied to the target absorbing body, receiving the photoacoustic signal in order to acquire a photoacoustic image, applying a critical value to pixels corresponding to microbubbles in the ultrasonic image in order to generate a mask image, and removing a background noise generated from a non-target absorbing body, while maintaining the target absorbing body, from the photoacoustic image using the mask image.

### CROSS-REFERENCE TO RELATED APPLICATIONS

This application claims priority under 35 U.S.C. § 119 to Korean Patent Application No. 10-2016-0146551 filed on Nov. 4, 2016 in the Korean Intellectual Property Office, the disclosure of which is incorporated herein by reference in its entirety.

### BACKGROUND OF THE INVENTION

### Field of the Invention

The present invention relates to a system and method for removing a background noise from a photoacoustic image, and more particularly to a system and method for removing a background noise from a photoacoustic image in order to improve accuracy of disease diagnosis using such a photoacoustic image.

### Description of the Related Art

A photoacoustic signal is an acoustic signal generated during a thermal expansion process occurring when a laser is applied to a biological tissue and the biological tissue absorbs the energy of the laser applied thereto. This signal has an ultrasonic frequency in a band ranging from several MHz to several tens of MHz. Consequently, the photoacoustic signal may be received by an ultrasonic probe, and various signal processing algorithms may be applied to the received signal in order to form an image.

The basic principle of a photoacoustic image will be described in more detail. A biological tissue is composed of different kinds of molecular tissues. A specific biological tissue has different laser absorption rates depending on the wavelength of the laser applied thereto.

For example, when a laser having a wavelength of 550 nm is applied to a human body, a hemoglobin component absorbs the energy of the laser having the above-specified wavelength better than other biological tissues. When a laser having a wavelength of 920 nm is applied to the human body, fat has the maximum degree of absorption. Taking advantage of this phenomenon, it is possible to enable a specific biological tissue to be imaged to more effectively absorb the energy of a laser applied thereto than peripheral tissues. Variation in a laser energy absorption rate on a per-biological-tissue basis becomes an important factor for deciding on the magnitude of a photoacoustic signal generated on a per-biological-tissue basis.

There is a necessity for developing a photoacoustic imaging technique on the above theoretical basis that is capable of acquiring a detailed image of an actual region to be measured.

### PRIOR ART DOCUMENT

### Patent Document

(Patent Document 1) Korean Registered Patent No. 10-1298935 (registered on Aug. 16, 2013 and entitled “Method and apparatus for producing ultrasound images and photoacoustic images”)

### SUMMARY OF THE INVENTION

It is an object of the present invention to provide a technical means for generating a mask image using an ultrasonic image acquired from a target absorbing body having a multi-modal microbubble contrast agent injected thereinto and removing a background noise, generated from a non-target absorbing body, from a photoacoustic image using the mask image.

In accordance with an aspect of the present invention, the above and other objects can be accomplished by the provision of a method of removing a background noise from a photoacoustic image, the method including applying an ultrasonic signal to a target absorbing body having a multi-modal microbubble contrast agent injected thereinto and receiving the ultrasonic signal reflected by the target absorbing body in order to acquire an ultrasonic image, when a photoacoustic signal is generated from the target absorbing body having the multi-modal microbubble contrast agent injected thereinto as the result of absorbing a laser pulse applied to the target absorbing body, receiving the photoacoustic signal in order to acquire a photoacoustic image, applying a critical value to pixels corresponding to microbubbles in the ultrasonic image in order to generate a mask image, and removing a background noise generated from a non-target absorbing body, while maintaining the target absorbing body, from the photoacoustic image using the mask image.

The method may further include, before the step of generating the mask image, improving a signal-to-noise ratio (SNR) and a contrast-to-noise ratio (CNR) through signal processing performed to increase the intensity of the ultrasonic signal generated by the multi-modal microbubble contrast agent such that the intensity of the ultrasonic signal is higher than the intensity of a photoacoustic signal generated from the non-target absorbing body and from the background of the target absorbing body.

The step of increasing the intensity of the ultrasonic signal may include tracking the ultrasonic signal applied to the target absorbing body using at least one selected from among a pulse inversion image, an image using a coded excitation technique using at least one selected from among chirp, Golay code, and Barker code, and a harmonic image.

The step of generating the mask image may include masking a region that is larger than the position of the microbubbles generated from the multi-modal microbubble contrast agent injected into the target absorbing body in order to prevent the loss of the photoacoustic signal.

The step of applying the critical value to the ultrasonic image in order to generate the mask image may include binarizing the ultrasonic image into 1 or 0 based on a predetermined threshold in order to generate the mask image and masking the photoacoustic image using the generated mask image.

The step of removing the background noise from the photoacoustic image may include multiplying the photoacoustic image by the mask image in order to acquire a photoacoustic image from which the background noise has been removed.

The step of generating the mask image may include repeating the receiving the ultrasonic image, acquiring a plurality of frame images of the target absorbing body from ultrasonic images received over time, and accumulating phase-shifted signals of the microbubbles generated in N frame images beginning with a first frame image, among the acquired frame images, in order to acquire an ultrasonic image.

In accordance with another aspect of the present invention, there is provided a system for removing a background noise from a photoacoustic image, the system including a probe for sequentially emitting an ultrasonic signal and a laser pulse to a target absorbing body having a multi-modal microbubble contrast agent injected thereinto and receiving an ultrasonic signal and a photoacoustic signal generated by the target absorbing body, an original image acquisition unit for acquiring an ultrasonic image and a photoacoustic image from the ultrasonic signal and the photoacoustic signal received by the probe, a mask image generation unit for applying a critical value to pixels of microbubbles in the ultrasonic image in order to generate a mask image, a noise removal unit for removing a background noise from the photoacoustic image using the mask image, and a display unit for displaying the photoacoustic image from which the background noise has been removed.

The mask image generation unit may improve a signal-to-noise ratio (SNR) and a contrast-to-noise ratio (CNR) through signal processing performed to increase the intensity of the ultrasonic signal generated by the multi-modal microbubble contrast agent such that the intensity of the ultrasonic signal is higher than the intensity of a photoacoustic signal generated from a non-target absorbing body and from the background of the target absorbing body, before generating the mask image.

The mask image generation unit may track the ultrasonic signal applied to the target absorbing body using at least one selected from among a pulse inversion image, an image using a coded excitation technique using at least one selected from among chirp, Golay code, and Barker code, and a harmonic image in order to increase the intensity of the ultrasonic signal.

The mask image generation unit may mask a region that is larger than the position of microbubbles generated from the multi-modal microbubble contrast agent injected into the target absorbing body in order to prevent the loss of the photoacoustic signal.

The mask image generation unit may binarize the ultrasonic image into 1 or 0 based on a predetermined threshold in order to generate the mask image and may mask the photoacoustic image using the generated mask image.

The noise removal unit may multiply the photoacoustic image by the mask image in order to acquire a photoacoustic image from which the background noise has been removed.

The mask image generation unit may repeat a process of receiving the ultrasonic image, may acquire a plurality of frame images of the target absorbing body from ultrasonic images received over time, and may accumulate phase-shifted signals of the microbubbles generated in N frame images beginning with a first frame image, among the acquired frame images, in order to acquire a pixel-wise displacement ultrasonic image.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram schematically showing a system for removing a background noise from a photoacoustic image according to an embodiment of the present invention;

FIG. 2 is a flowchart showing a method of removing a background noise from a photoacoustic image according to an embodiment of the present invention;

FIG. 3 is a view showing an ideal diagnosis method using an ultrasonic image and a photoacoustic image according to an embodiment of the present invention;

FIG. 4 is a flowchart showing a pixel-wise displacement technique according to an embodiment of the present invention;

FIG. 5 is a view showing an experimental environment configured to demonstrate the system for removing the background noise from the photoacoustic image according to the embodiment of the present invention; and

FIGS. 6A to 6D are photographs showing simulation results based on an experiment for removing a background noise from a photoacoustic image.

### DETAILED DESCRIPTION OF THE INVENTION

Prior to a description of embodiments of the present invention, problems afflicting a conventional photoacoustic imaging method will be examined, and then a technical means adopted by the embodiments of the present invention in order to solve these problems will be briefly introduced.

In a photoacoustic imaging technique, a laser signal is transmitted so as to be absorbed by a specific molecule (hereinafter, also referred to as a “target absorbing body”), and a photoacoustic signal generated from the target absorbing body that has absorbed the energy of the transmitted laser is received to form an image. In order to generate a photoacoustic signal, it is necessary to emit a laser signal having a specific wavelength such that the target absorbing body absorbs the energy of the emitted laser.

Ideally, only the target absorbing body should absorb the energy of the laser in order to generate a photoacoustic signal. In actuality, however, a photoacoustic signal is generated from a non-target absorbing body or from the vicinity of the target absorbing body even though the magnitude of the photoacoustic signal is relatively small.

The photoacoustic signal generated from the non-target absorbing body or from the background of the target absorbing body appears as a strong background noise through the entirety of a photoacoustic image. Moreover, this problem frequently occurs even when a contrast agent that is injected into a biological tissue from the outside or a target contrast agent is used.

For this reason, it is necessary to provide a solution for accurately separating a target absorbing body from the background of the target absorbing body and distinguishing between the target absorbing body and a non-target absorbing body.

Therefore, embodiments of the present invention propose a technical means for accurately separating a target absorbing body from the background of the target absorbing body and distinguishing between the target absorbing body and a non-target absorbing body.

Hereinafter, embodiments of the present invention will be described in detail with reference to the accompanying drawings. In the following specification and the accompanying drawings, a detailed description of known functions or structures incorporated herein will be omitted when the same may make the subject matter of the present invention rather unclear. Also, it is to be noted that the same constituent elements are designated by the same reference numerals even when they are depicted in different drawings.

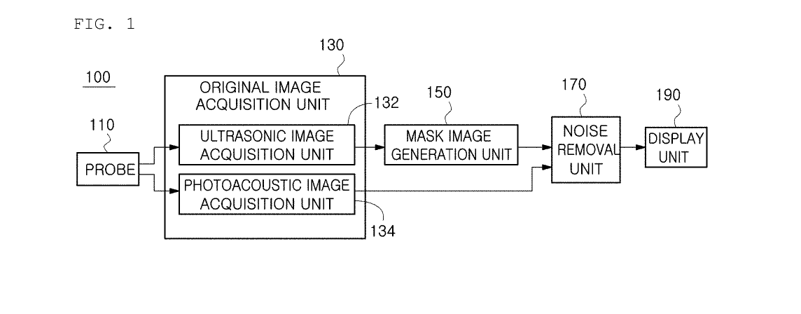

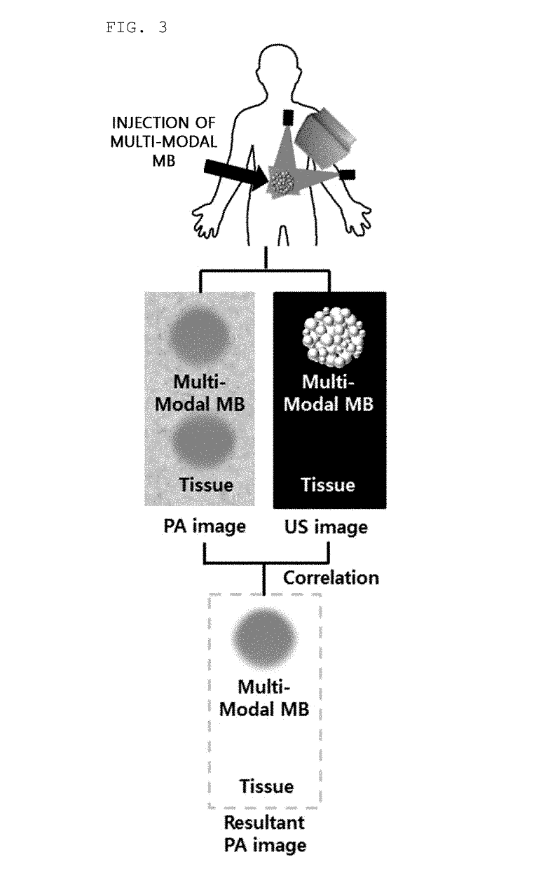

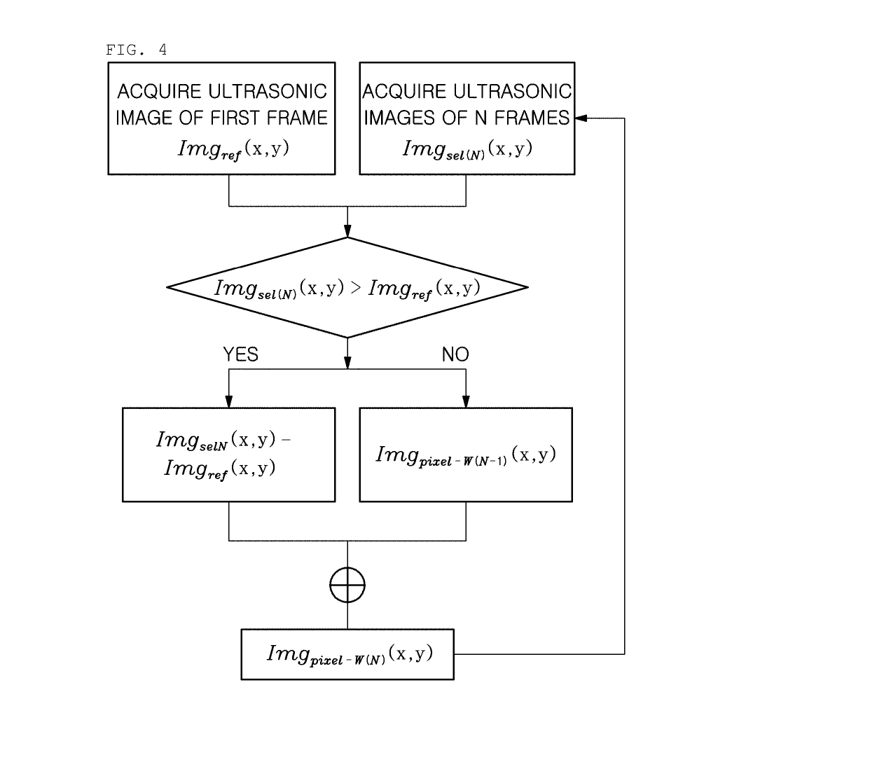

FIG. 1 is a block diagram schematically showing a system for removing a background noise from a photoacoustic image according to an embodiment of the present invention, FIG. 3 is a view showing an ideal diagnosis method using an ultrasonic image and a photoacoustic image according to an embodiment of the present invention, and FIG. 4 is a flowchart showing a pixel-wise displacement technique according to an embodiment of the present invention.

Referring to FIG. 1, a system 100 for removing a background noise from a photoacoustic image according to an embodiment of the present invention includes a probe 110, an original image acquisition unit 130, a mask image generation unit 150, a noise removal unit 170, and a display unit 190.

The probe 110 sequentially emits an ultrasonic signal and a laser pulse to a target absorbing body having a multi-modal microbubble contrast agent injected thereinto and receives an ultrasonic signal and a photoacoustic signal generated by the target absorbing body. That is, the probe 110 may emit an ultrasonic signal to an object, and may receive the ultrasonic signal reflected by the object. In addition, the probe 110 may emit light to the object, and may receive a photoacoustic signal generated by the object.

Here, multi-modal microbubbles, which serve as a contrast agent for an ultrasonic image together with a photoacoustic image, are injected into a biological tissue to be diagnosed, i.e. a target absorbing body, as shown in FIG. 3.

The original image acquisition unit 130 includes an ultrasonic image acquisition unit 132 and a photoacoustic image acquisition unit 134. Specifically, the ultrasonic image acquisition unit 132 receives an ultrasonic signal that is applied to a target absorbing body having a target absorbing body injected thereinto and is then reflected by the target absorbing body in order to generate an ultrasonic image (indicated as “US image” in FIG. 3). To this end, the ultrasonic signal may undergo beam focusing, envelope detection, and signal processing.

In addition, the ultrasonic image acquisition unit 132 may improve a signal-to-noise ratio (SNR) and a contrast-to-noise ratio (CNR) through signal processing performed to increase the intensity of an ultrasonic signal generated by the multi-modal microbubble contrast agent such that the intensity of the ultrasonic signal is higher than the intensity of a photoacoustic signal generated from a non-target absorbing body and from the background of the target absorbing body.

In order to increase the intensity of the ultrasonic signal, the SNR and the CNR may be improved while tracking the ultrasonic signal applied to the target absorbing body using at least one selected from among a pulse inversion image, an image using a coded excitation technique using at least one selected from among chirp, Golay code, and Barker code, and a harmonic image.

The photoacoustic image acquisition unit 134 receives an ultrasonic signal generated from a target absorbing body having a multi-modal microbubble contrast agent injected thereinto as the result of absorbing a laser pulse applied to the target absorbing body in order to generate a photoacoustic image (indicated as “PA image” in FIG. 3). To this end, the ultrasonic signal may undergo beam focusing, envelope detection, and signal processing.

The mask image generation unit 150 applies a critical value to the pixels of microbubbles in the ultrasonic image generated by the ultrasonic image acquisition unit 132 that have greater pixel values than other tissues in order to generate a mask image. To this end, a phenomenon in which the mask image generated using the ultrasonic image indicates only the position of the microbubbles is used.

In order to prevent the loss of the photoacoustic signal, a region that is larger than the position of the microbubbles generated from the multi-modal microbubble contrast agent injected into the target absorbing body may be masked.

The ultrasonic image is binarized into 1 or 0 based on a predetermined threshold in order to mask the ultrasonic image.

Particularly, in order to generate a mask image, the ultrasonic image acquisition unit 132 repeatedly receives ultrasonic images, acquires a plurality of frame images of the target absorbing body from ultrasonic signals received over time, and accumulates phase-shifted signals of the microbubbles generated in N frame images beginning with the first frame image, among the acquired frame images, in order to acquire an error-compensated ultrasonic image. FIG. 4 shows such a pixel-wise displacement technique.

That is, in a general tissue, the same signal may be received from a plurality of frame images. In contrast, microbubbles move and are phase-shifted over time, whereby the signal received from each frame over time is not uniform. In the case in which a frame is acquired only at a specific time, therefore, an error occurs for the above-mentioned reason. In order to compensate for this, accumulated images are generated. Consequently, it is possible to widely set the position of the microbubbles by accumulating a plurality of frames acquired over time with respect to the microbubbles that are not uniformly received, whereby it is possible to compensate for an error in a signal. Therefore, it is possible to image a photoacoustic signal without loss.

The noise removal unit 170 removes a background noise, generated from the non-target absorbing body and from the background of the target absorbing body, from the photoacoustic image using the mask image. To this end, the photoacoustic image is multiplied by the mask image, whereby it is possible to acquire a photoacoustic image from which the background noise has been removed. Since the ultrasonic image is binarized into 1 or 0 based on a predetermined threshold, the remainder of the image is removed using the mask image, excluding a photoacoustic image corresponding to the ultrasonic image, whereby the background noise is removed from the photoacoustic image.

The display unit 190 outputs the photoacoustic image from which the background noise has been removed by the noise removal unit 170.

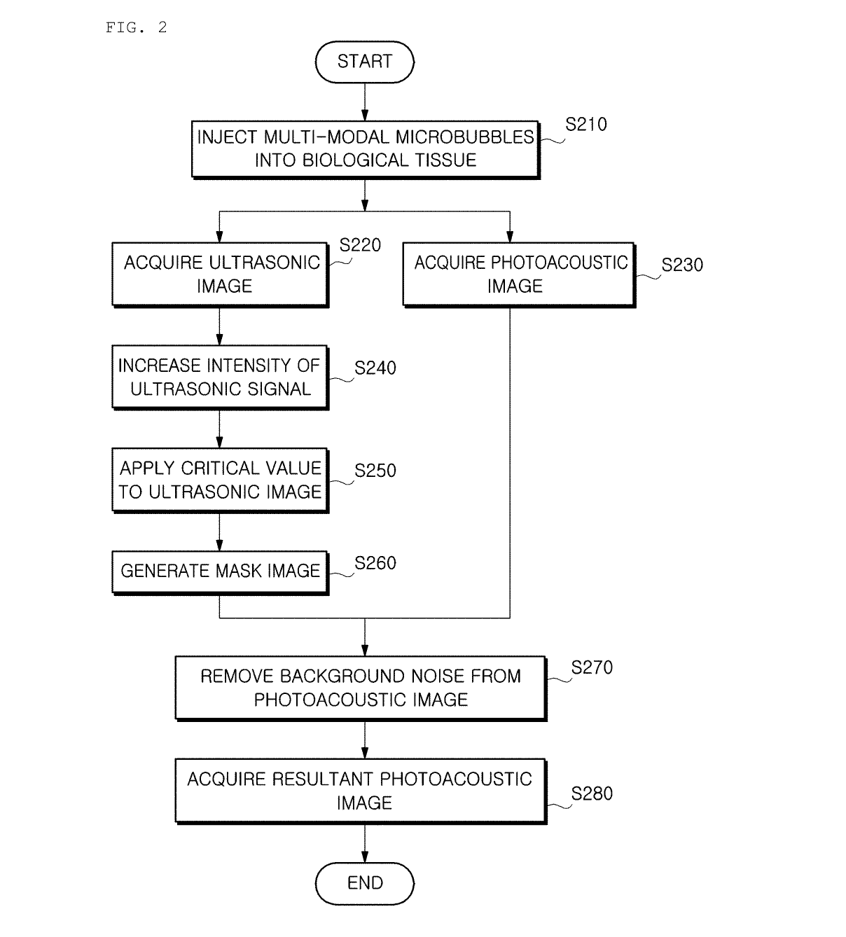

Hereinafter, a method of removing a background noise from a photoacoustic image according to an embodiment of the present invention will be described in detail with reference to FIG. 2. FIG. 2 is a flowchart showing a method of removing a background noise from a photoacoustic image according to an embodiment of the present invention.

Referring to FIG. 2, in order to remove a background noise from a photoacoustic image according to an embodiment of the present invention, a multi-modal microbubble contrast agent is injected into a biological tissue to be diagnosed (S210). That is, a multi-modal microbubble contrast agent is injected into a target absorbing body. Microbubbles are generated in the target absorbing body, into which the multi-modal microbubble contrast agent has been injected, over time.

Subsequently, an ultrasonic signal is applied to the target absorbing body, into which the multi-modal microbubble contrast agent has been injected, and the ultrasonic signal reflected by the target absorbing body is received to thus acquire an ultrasonic image (S220).

Subsequently, when a laser pulse is applied to the target absorbing body, into which the multi-modal microbubble contrast agent has been injected, the applied laser pulse is absorbed by the target absorbing body, and an ultrasonic signal is generated, the ultrasonic signal is received to acquire a photoacoustic image (S230). Here, the target absorbing body to which the laser pulse has been applied is identical to the target absorbing body to which the ultrasonic signal was applied at step S220.

Subsequently, a signal-to-noise ratio (SNR) and a contrast-to-noise ratio (CNR) are improved through signal processing performed to increase the intensity of an ultrasonic image generated by the multi-modal microbubble contrast agent such that the intensity of the ultrasonic image is higher than the intensity of an ultrasonic signal generated from a non-target absorbing body and from the background of the target absorbing body (S240).

In order to increase the intensity of the ultrasonic signal, the SNR and the CNR may be improved while tracking the ultrasonic signal applied to the target absorbing body using at least one selected from among a pulse inversion image, an image using a coded excitation technique using at least one selected from among chirp, Golay code, and Barker code, and a harmonic image.

Subsequently, a critical value is applied to the pixels of microbubbles in the ultrasonic image generated at step S220 that have greater pixel values than other tissues (S250), whereby a mask image is generated (S260). To this end, a phenomenon in which the mask image generated using the ultrasonic image indicates only the position of the microbubbles is used.

In order to prevent the loss of the photoacoustic signal, a region that is larger than the position of microbubbles generated from the multi-modal microbubble contrast agent injected into the target absorbing body may be masked.

The ultrasonic image is binarized into 1 or 0 based on a predetermined threshold in order to generate a mask image

At the step of generating the mask image, as shown in FIG. 4, the process of receiving the ultrasonic image is repeated, a plurality of frame images of the target absorbing body is acquired from ultrasonic signals received over time, and phase-shifted signals of the microbubbles generated in N frame images beginning with the first frame image, among the acquired frame images, are accumulated to acquire an error-compensated ultrasonic image.

That is, in a general tissue, the same signal may be received from a plurality of frame images. In contrast, microbubbles move and are phase-shifted over time, whereby the signal received from each frame over time is not uniform. In the case in which a frame is acquired only at a specific time, therefore, an error occurs for the above-mentioned reason. In order to compensate for this, accumulated images are generated. Consequently, it is possible to widely set the position of the microbubbles by accumulating a plurality of frames acquired over time with respect to the microbubbles that are not uniformly received, whereby it is possible to compensate for an error in a signal. Therefore, it is possible to image a photoacoustic signal without loss.

Subsequently, a background noise generated from the non-target absorbing body and from the background of the target absorbing body is removed from the photoacoustic image using the mask image (S270). To this end, the photoacoustic image is multiplied by the mask image, whereby it is possible to acquire a photoacoustic image from which the background noise has been removed. Since the ultrasonic image is binarized into 1 or 0 based on a predetermined threshold, the remainder of the image is removed using the mask image, excluding a photoacoustic image corresponding to the ultrasonic image, whereby the background noise is removed from the photoacoustic image.

Consequently, a resultant photoacoustic image from which the background noise has been removed is acquired (S280).

In the present invention, as described above, an ultrasonic image is acquired, a pixel value based on the magnitude of a received signal is applied to the acquired ultrasonic image in order to generate a mask, and a target absorbing body is distinguished from a non-target absorbing body and the background of the target absorbing body using the generated mask.

In addition, it is possible to compensate for a diagnosis error and to improve accuracy through the use of a photoacoustic image generation method that is characterized by removing a photoacoustic signal corresponding to a non-target absorbing body, rather than a target absorbing body, through contrast enhancement, unlike an image using a general photoacoustic contrast agent. Consequently, it is possible for a user to realize only an object to be diagnosed with higher contrast resolution.

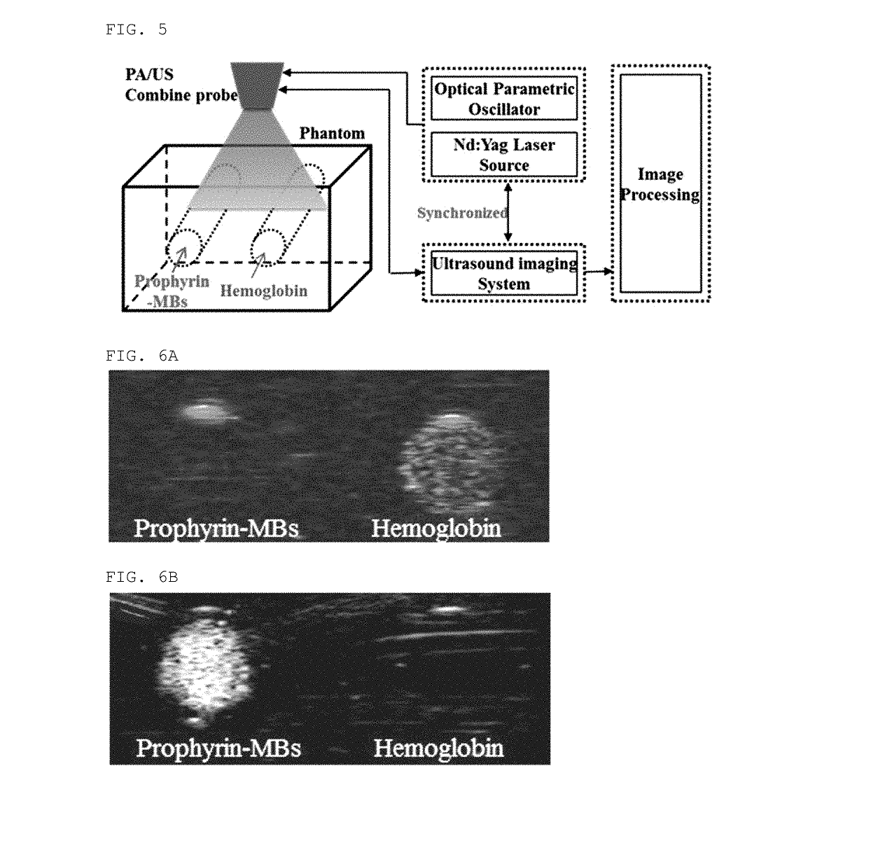

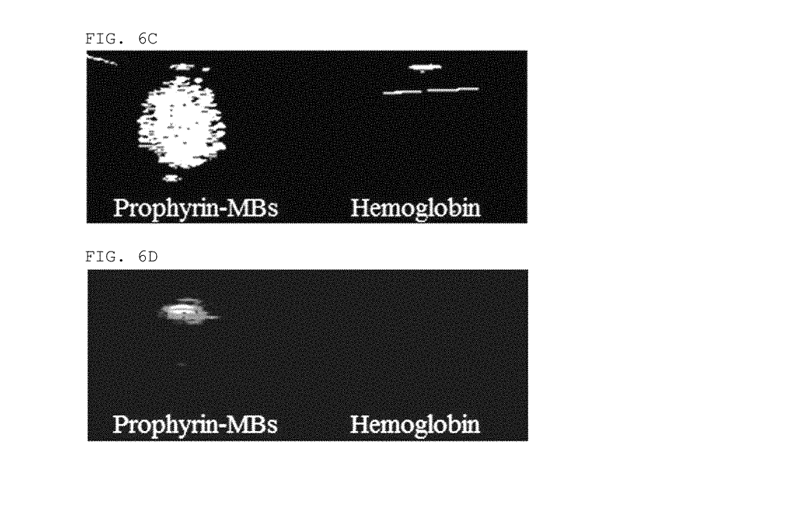

FIG. 5 is a view showing an experimental environment configured to demonstrate the system for removing the background noise from the photoacoustic image according to the embodiment of the present invention, and FIGS. 6A to 6D are photographs showing experimental results based on an experiment for removing a background noise from a photoacoustic image.

Referring to FIG. 5, in order to demonstrate the system for removing the background noise from the photoacoustic image according to the present invention, a target contrast agent, porphyrin-MBs, and a non-target material, hemoglobin, were injected into a tissue mimicking phantom, and an ultrasonic image and a photoacoustic image were acquired using a commercial ultrasonic system and a laser. The ultrasonic image was signal-processed in order to increase the intensity of a signal, and a critical value was applied to the pixels of the microbubbles that have greater pixel values than other tissues in order to generate a mask image. A background noise generated from the non-target absorbing body and from the background of the target absorbing body was removed from a photoacoustic image using the mask image.

FIG. 6A shows a photoacoustic image, FIG. 6B shows an ultrasonic image, FIG. 6C shows a mask image, and FIG. 6D shows a resultant photoacoustic image. It can be seen from FIGS. 6A to 6D that information about the position of a target is acquired and indicated using the ultrasonic image in order to image only a desired target contrast agent, porphyrin-MBs, from the photoacoustic image, whereby it is possible to remove a background noise from the photoacoustic image.

As is apparent from the above description, according to an embodiment of the present invention, a mask image is generated using an ultrasonic image acquired from a target absorbing body having a multi-modal microbubble contrast agent injected thereinto, and a background noise generated from a non-target absorbing body is removed from a photoacoustic image using the mask image, whereby it is possible to improve accuracy in disease diagnosis using the photoacoustic image.

That is, an ultrasonic image is acquired, a pixel value based on the magnitude of a received signal is applied to the acquired ultrasonic image in order to generate a mask, and a target absorbing body is distinguished from a non-target absorbing body and the background of the target absorbing body using the generated mask.

In addition, it is possible to compensate for a diagnosis error and to improve accuracy through the use of a photoacoustic image generation method that is characterized by removing a photoacoustic signal corresponding to a non-target absorbing body, rather than a target absorbing body, through contrast enhancement, unlike an image using a general photoacoustic contrast agent. Consequently, it is possible for a user to realize only an object to be diagnosed with higher contrast resolution.

Meanwhile, the embodiments of the present invention may be implemented as code that can be written in a computer-readable recording medium and thus read by a computer system. The computer-readable recording medium may be any type of recording device in which data that can be read by the computer system is stored.

Examples of the computer-readable recording medium include a ROM, a RAM, a CD-ROM, a magnetic tape, a floppy disk, and an optical data storage device. In addition, the computer-readable recording medium can be distributed over computer systems connected to a network so that computer-readable code is written thereto and executed therefrom in a distributed manner. Functional programs, code, and code segments to realize the present invention herein can be easily devised by programmers skilled in the art to which the present invention pertains.

While the present invention has been shown and described with particular reference to exemplary embodiments thereof, those skilled in the art will appreciate that the present invention may be embodied in specific forms other than those set forth herein without departing from the spirit and essential characteristics of the present invention. The disclosed embodiments are therefore to be construed in all aspects as illustrative and not restrictive. The scope of the present invention should be determined by reasonable interpretation of the appended claims, and all changes coming within the equivalency range of the invention are within the scope of the invention.

1. A method of removing a background noise from a photoacoustic image, the method comprising:
applying an ultrasonic signal to a target absorbing body having a multi-modal microbubble contrast agent injected thereinto and receiving the ultrasonic signal reflected by the target absorbing body in order to acquire an ultrasonic image;
when a photoacoustic signal is generated from the target absorbing body having the multi-modal microbubble contrast agent injected thereinto as a result of absorbing a laser pulse applied to the target absorbing body, receiving the photoacoustic signal in order to acquire a photoacoustic image;
applying a critical value to pixels corresponding to microbubbles in the ultrasonic image in order to generate a mask image; and
removing a background noise generated from a non-target absorbing body, while maintaining the target absorbing body, from the photoacoustic image using the mask image.

2. The method according to claim 1, further comprising, before the step of generating the mask image, improving a signal-to-noise ratio (SNR) and a contrast-to-noise ratio (CNR) through signal processing performed to increase an intensity of the ultrasonic signal generated by the multi-modal microbubble contrast agent such that the intensity of the ultrasonic signal is higher than an intensity of a photoacoustic signal generated from the non-target absorbing body and from a background of the target absorbing body.

3. The method according to claim 2, wherein the step of increasing the intensity of the ultrasonic signal comprises tracking the ultrasonic signal applied to the target absorbing body using at least one selected from among a pulse inversion image, an image using a coded excitation technique using at least one selected from among chirp, Golay code, and Barker code, and a harmonic image.

4. The method according to claim 1, wherein the step of generating the mask image comprises masking a region that is larger than a position of the microbubbles generated from the multi-modal microbubble contrast agent injected into the target absorbing body in order to prevent a loss of the photoacoustic signal.

5. The method according to claim 1, wherein the step of applying the critical value to the ultrasonic image in order to generate the mask image comprises binarizing the ultrasonic image into 1 or 0 based on a predetermined threshold in order to generate the mask image and masking the photoacoustic image using the generated mask image.

6. The method according to claim 5, wherein the step of removing the background noise from the photoacoustic image comprises multiplying the photoacoustic image by the mask image in order to acquire a photoacoustic image from which the background noise has been removed.

7. The method according to claim 1, wherein the step of generating the mask image comprises repeating the receiving the ultrasonic image, acquiring a plurality of frame images of the target absorbing body from ultrasonic images received over time, and accumulating phase-shifted signals of the microbubbles generated in N frame images beginning with a first frame image, among the acquired frame images, in order to acquire an ultrasonic image.

8. A computer-readable recording medium having a program for allowing a computer to execute the method according to claim 1 recorded therein.

9. A system for removing a background noise from a photoacoustic image, the system comprising:
a probe for sequentially emitting an ultrasonic signal and a laser pulse to a target absorbing body having a multi-modal microbubble contrast agent injected thereinto and receiving an ultrasonic signal and a photoacoustic signal generated by the target absorbing body;
an original image acquisition unit for acquiring an ultrasonic image and a photoacoustic image from the ultrasonic signal and the photoacoustic signal received by the probe;
a mask image generation unit for applying a critical value to pixels of microbubbles in the ultrasonic image in order to generate a mask image;
a noise removal unit for removing a background noise from the photoacoustic image using the mask image; and
a display unit for displaying the photoacoustic image from which the background noise has been removed.

10. The system according to claim 9, wherein the mask image generation unit improves a signal-to-noise ratio (SNR) and a contrast-to-noise ratio (CNR) through signal processing performed to increase an intensity of the ultrasonic signal generated by the multi-modal microbubble contrast agent such that the intensity of the ultrasonic signal is higher than an intensity of a photoacoustic signal generated from a non-target absorbing body and from a background of the target absorbing body, before generating the mask image.

11. The system according to claim 9, wherein the mask image generation unit tracks the ultrasonic signal applied to the target absorbing body using at least one selected from among a pulse inversion image, an image using a coded excitation technique using at least one selected from among chirp, Golay code, and Barker code, and a harmonic image in order to increase the intensity of the ultrasonic signal.

12. The system according to claim 9, wherein the mask image generation unit masks a region that is larger than a position of microbubbles generated from the multi-modal microbubble contrast agent injected into the target absorbing body in order to prevent a loss of the photoacoustic signal.

13. The system according to claim 9, wherein the mask image generation unit binarizes the ultrasonic image into 1 or 0 based on a predetermined threshold in order to generate the mask image and masks the photoacoustic image using the generated mask image.

14. The system according to claim 13, wherein the noise removal unit multiplies the photoacoustic image by the mask image in order to acquire a photoacoustic image from which the background noise has been removed.

15. The system according to claim 9, wherein the mask image generation unit repeats a process of receiving the ultrasonic image, acquires a plurality of frame images of the target absorbing body from ultrasonic images received over time, and accumulates phase-shifted signals of the microbubbles generated in N frame images beginning with a first frame image, among the acquired frame images, in order to acquire a pixel-wise displacement ultrasonic image.
